(19) United States
(12) Patent Application Publication
(10) Pub. No.: US 2005/0191758 A1
(43) Pub. Date: Sep. 1, 2005

(54) Soil test box

(76) Inventors: John Pether, Kings Langley (AU); David Sharman, Plant City, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/647,998

(22) Filed: Aug. 26, 2003

Related U.S. Application Data
(60) Provisional application No. 60/406,408, filed on Aug. 26, 2002.

(51) Int. Cl. G01N 33/00; G01N 1/00

(52) U.S. Cl. 436/174; 422/68.1

(57) ABSTRACT

A soil test apparatus (1) for testing products in subterranean soil installations. The apparatus includes a container (3) for receiving soil (15) and a product (13) to be buried therein for testing. The container includes a base (5), sides (4, 7) and a top opening adapted directly or indirectly to receive a load. At least one of the sides is configured to permit some movement or deformation under application of the load to the soil in the container so as to simulate larger scale installation conditions. A method of testing products in subterranean soil installations using the soil test apparatus comprises setting up the apparatus to permit some movement or deformation of at least one of the sides (4, 7) under predetermined load conditions. The container is partially filled with soil (15). The product (13) is positioned within the container. The product is buried by the addition of further soil (15). The load is applied to the soil via the top opening of the container. An effect of the load on the product, the container, or the soil is measured.

### CROSS-REFERENCE TO RELATED APPLICATION

This application claims the benefit of U.S. Provisional Application No. 60/406,408, filed Aug. 26, 2002, the entirety of which is hereby incorporated by reference.

### BACKGROUND OF THE INVENTION

1. Field of the Invention

The present invention relates in certain embodiments to an apparatus and associated method for workshop or laboratory testing of products in simulated subterranean soil installations.

Preferred embodiments have been developed for specific use in the testing of pipes and more particularly concrete pipes, and will be described hereinafter with reference to this field of use. However, it will be appreciated that the invention is readily adapted for use in simulated subterranean soil testing of other products.

2. Description of the Related Art

Product performance and properties are two of the most important features of building products, particularly products that are to be installed underground which makes periodic inspection and replacement difficult and expensive. Ideally, product samples of this kind, such as water pipes, should be tested under actual conditions prior to mass production to ensure that they meet established performance standards. However, testing under actual conditions can be very difficult, time consuming and expensive, particularly when the product is to be installed underground.

Up until now testing standards have focused on product performance in the laboratory. Tests that try to address the in-situ performance have necessitated the use of soil test boxes that are the size of an average room. The reasons for the cumbersome size of a traditional soil test box is that it was considered essential that there be a minimum volume of surrounding soil material (usually expressed in terms of multiples of the diameter of the pipe to be tested) to minimise the boundary effects of the container.

Because it is both costly and time consuming to build and maintain these soil test boxes, most companies need to send samples to universities, research institutes or specialist testing laboratories, which are similarly bound by the time and cost constraints associated with these large scale facilities.

The need to contract out work of this kind impacts significantly on development costs. It also significantly slows the development process, due to testing costs and perhaps more importantly due to the limited availability of suitable testing facilities.

It is an object of the present invention to overcome or ameliorate one or more of the disadvantages of the prior art, or at least to provide a useful alternative.

### SUMMARY OF THE INVENTION

According to a first aspect of the invention, there is provided a soil test apparatus for testing products in subterranean soil installations, the system including:
a container for receiving soil and said product to be buried therein,
said container having a base, side portions and a top opening adapted for directly or indirectly receiving a load, for example, via a loading application means,
wherein at least a part of said side portions is configured to permit some movement or deformation under application of said load to the soil in the container.

Preferably the movement or deformation is such that in use the test approximately simulates larger scale installation conditions.

The term soil is used herein to refer to soil, sand, gravel or any other suitable substitute filler material that will function in a manner generally indicative of the subterranean conditions to be simulated.

In a preferred form of the invention that is specifically configured for testing longitudinal hollow products such as pipes, the container is generally rectangular in plan view having two spaced side portions that in use are generally parallel to the axis of the pipe, and two opposing end portions that in use extend generally transverse to the pipe axis, wherein two of said side portions are configured to permit movement or deformation under application of a load to the soil therein.

Preferably, the side portions are configured to permit resilient movement or deformation under pressure transferred from the compacted soil in the test box. In a preferred form, these side portions comprise a rigid wall section that is configured to permit outward movement against a resilient biasing means. In one preferred form, the resilient biasing means comprise a leaf spring arrangement secured to a fixed frame that forms part of the container structure.

In another embodiment, one of said side portions is configured to permit movement or deformation under application of a load to the soil therein, the other said portion being fixed. In this embodiment, the soil test box is preferably laterally fixed in relation to the facility that applies the load.

In another embodiment, these side portions are fixed but deformable by virtue of their structure and/or the materials from which they are made.

Desirably, the apparatus also includes viewing windows at convenient locations. In the embodiment configured for testing pipes, the viewing windows are provided in the fixed rigid end portions. In this embodiment, strategically placed openings may also be provided for access to the interior of the pipe during testing to facilitate deflection measurement, photographic recordal, and the like.

Optionally, the apparatus further includes water introduction means for varying the soil moisture conditions and/or means for heating or cooling the contents of the container.

The relative size of the product when compared to the width of the soil test box can be determined by skilled workers in this field.

According to a second aspect of the invention, there is provided a method of testing products in subterranean soil installations using the apparatus of the first aspect, the method comprising the steps of:
setting up the apparatus to permit some movement or deformation of a part of said side portions under certain load conditions;
partially filling the container with a selected soil material or substitute therefor;
positioning the product within the container;
burying the product by the addition of more soil material;
applying a direct or indirect load to the soil via the top opening of the container; and
measuring the effect of the soil loading on the product.

The method may also include the optional steps of measuring product stress, strain, deflection, soil stress, movement of the flexible end portions and/or visually monitoring the product during testing.

Desirably, both the apparatus and method of the invention may include the use of liners or other means of minimizing side wall friction, such as polyethylene lining sheets lubricated with grease. The selection and application of appropriate linings and/or lubricant if required, can be determined by skilled workers in this field.

### BRIEF DESCRIPTION OF THE DRAWINGS

A preferred embodiment of the invention will now be described, by way of example only, with reference to the accompanying drawings in which:

FIG. 1 is a schematic plan view of a flexible walled miniature soil test box in accordance with a first embodiment of the invention;

FIG. 2 is a schematic front view of the soil test box shown in FIG. 1;

FIG. 3 is a schematic end view of the soil test box shown in the previous figures;

FIG. 4 is a flow chart showing a method of measuring the test article performance in an installed condition within the soil test box of the previous figures; and

FIG. 5 is a flow chart indicating a method of using the flexible miniature soil test box according to one embodiment of the invention.

### DETAILED DESCRIPTION OF THE PREFERRED EMBODIMENTS

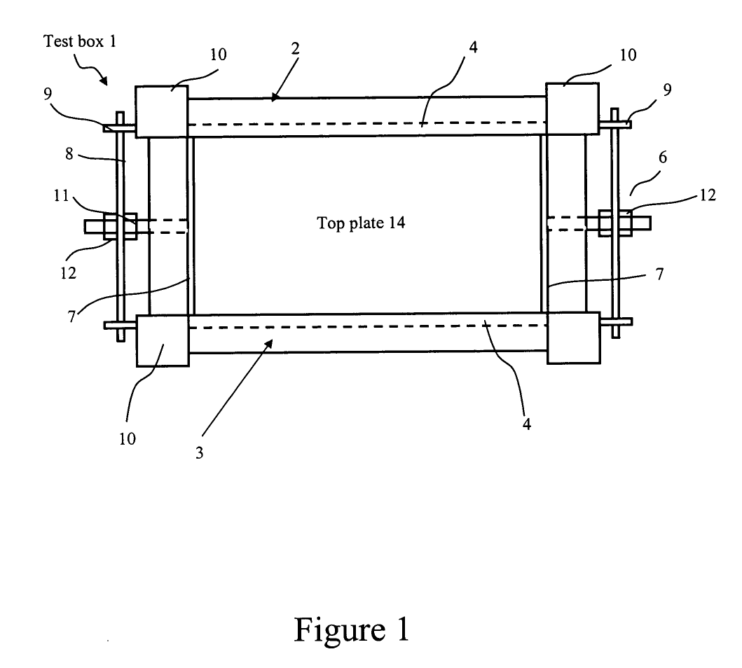

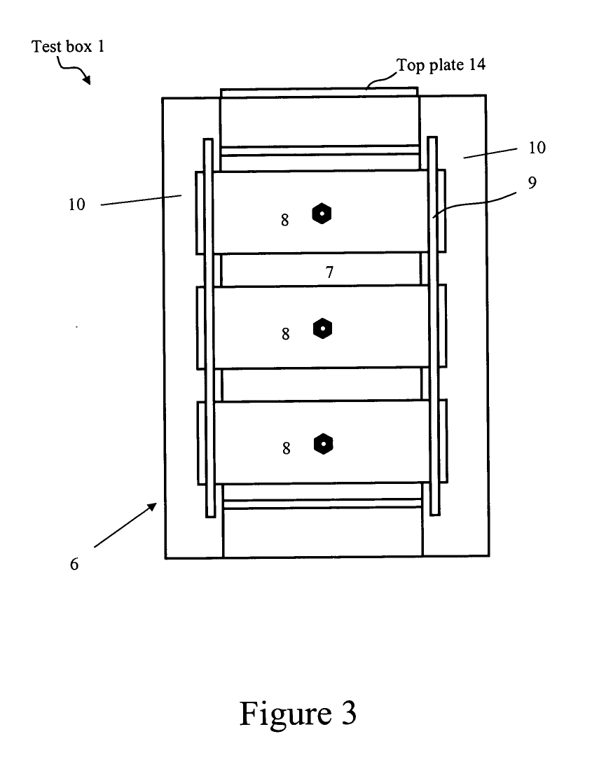

Referring to FIGS. 1-3, one embodiment of the invention provides a soil test box 1 specifically configured for use in testing pipes. The box includes a rigid external frame 2 that defines the exterior of the container 3. Fixed end portions in the form of end plates 4 and a fixed base portion 5 are secured to the frame. Resiliently movable end portions shown generally at 6 form the remaining two walls of the container 3. These end portions include end plates 7, which are ideally made of a material such as steel or aluminum, are mounted with the frame 2 via an intermediate spring mechanism. This spring mechanism includes a set of spaced apart generally parallel leaf springs 8, each mounted at its ends with flanges 9 extending outwardly from the respective corner posts 10 of the frame. The end plates 7 are then connected with the leaf springs 8 by means of respective connecting rods 11 extending from the rigid end plates 7. The connecting rods are bolted securely to the leaf spring 8 by respective pairs of lock nuts 12, as shown.

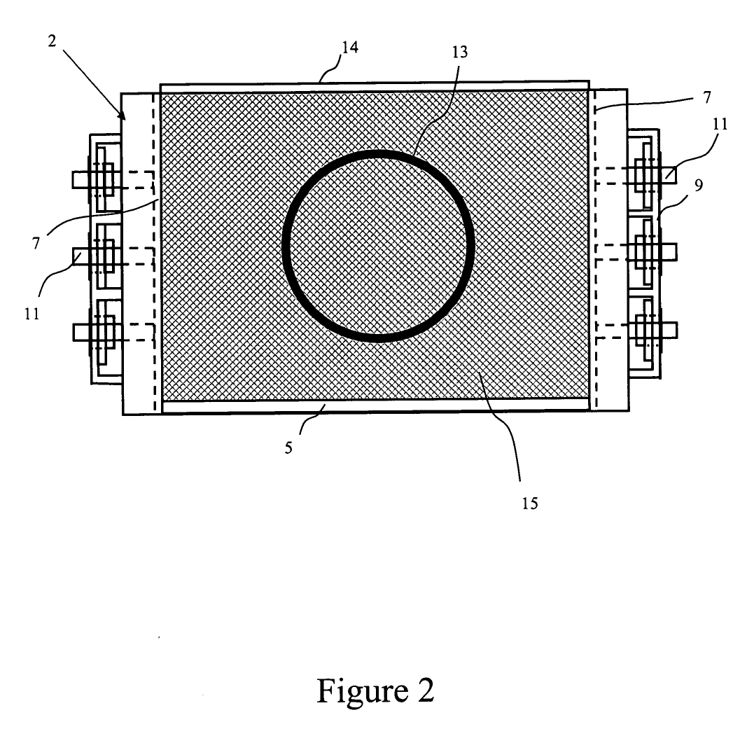

In a preferred embodiment, the fixed rigid side plates 4 may be made of a transparent material such as plexiglass which if necessary can be supported by a metal lattice (not shown) secured to the frame 2. In other embodiments, the wall may be steel or a similar structural material, with only a smaller viewing window. In yet another embodiment, the side plates 4 include an opening for accessing the interior of a test article, such as the pipe 13 shown in FIG. 2. An optional top plate 14 may also be provided as part of the test box.

In use, the test box is set up with a predetermined number of leaf springs 8 in the form of strips of spring steel of a known spring rating which are then connected to the movable end plates 7 as shown. The box is then partially filled with a predetermined amount of filler 15, which can be any suitable material such as soil, sand, gravel, or the like. The test article such as pipe 13 is then connected to various sensors (not shown) to measure performance parameters such as load bearing capacity and deflection either continuously or at predetermined intervals during the test, and to create a corresponding data log for subsequent analysis.

The pipe 13 is then placed into the partially filled test box 1, and additional filler is added to a pre-set level. The optional top plate 14 is then placed onto the filler. Whether the top plate is required will be determined by the proposed method of applying the test loads.

In a preferred form, the loaded test box with top plate 14 attached is then positioned under a suitable loading apparatus such as a “Universal” testing machine, which applies a load to the top plate. Depending on the requirements of the particular test, a load could similarly be applied by using a weight on the top plate, by applying pressure via an inflatable diaphragm, or by other suitable mains. Alternatively, the soil box could be configured such that a load plate of the load-applying machine fits directly into the soil box making, the separate top plate 14 unnecessary.

As the load is applied to the contents of the container, the filler modulus changes and the test article deforms as part of the load is transferred horizontally. This also causes the end plates 7 to move slightly outwardly against the known biasing force of the springs 8.

The load placed on the test article 13 is measured, as is (optionally) the deflection of the test article within the test box. After a predetermined load and period of time, the test article is removed from the test box for evaluation and the results are analysed.

Optionally, the test box can be used for other testing procedures. For example, the test box could be filled with a predetermined amount of moisture, and the contents frozen by using, for example, cooling coils, a cooling jacket or a freezer, in order to test performance under freeze-thaw conditions. Other embodiments of the invention may include water delivery means (not shown) for varying the moisture content of the filler material during the test. The apparatus may also include integral heating and/or cooling systems.

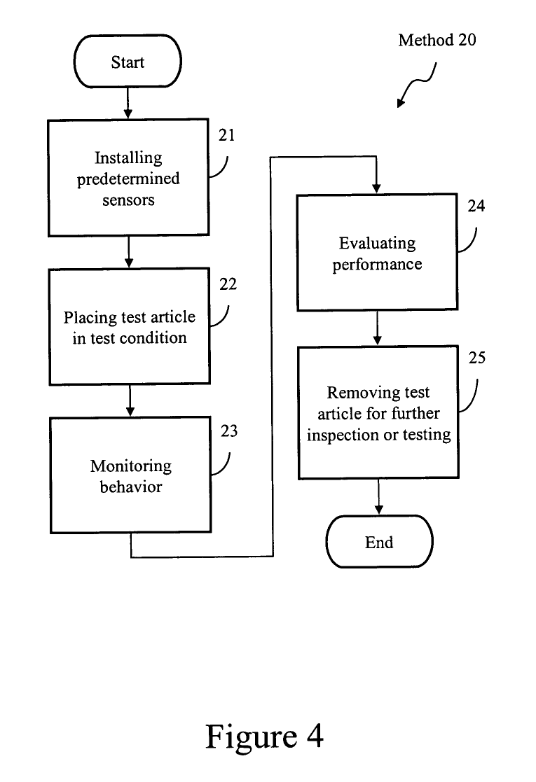

Referring next to FIG. 4, there is shown a schematic flow chart of the basic procedures involved in measuring the performance of the test article in the installed condition using the test box in accordance with one embodiment of the invention.

In a preferred form, the method of measuring 20 includes the following steps:

Step 21—Installing predetermined sensors. In this step, a predetermined array of sensors for measuring compaction results, such as strain, deflection, etc., are installed on test article 13 (e.g., pipe, column, footing, pits etc.).

Step 22—Placing test article in test condition. In this step, the test article 13 is placed into test box 1 under simulated installed conditions.

Step 23—Monitoring behaviour. In this step, the behaviour of the test article is monitored under installed conditions utilizing the sensors installed in step 21.

Step 24—Evaluating performance. In this step, the performance of test article 13 is evaluated from data generated under installed conditions and recorded in a data log. Depending on filler 15 or test conditions, this data could provide information on the effects of soil compaction, moisture or freeze-thaw on product performance.

Step 25—Removing test article for further inspection or testing. In this step, the test article 13 is optionally removed from the installed condition in the test box for further inspection or testing to analyse physical properties if necessary.

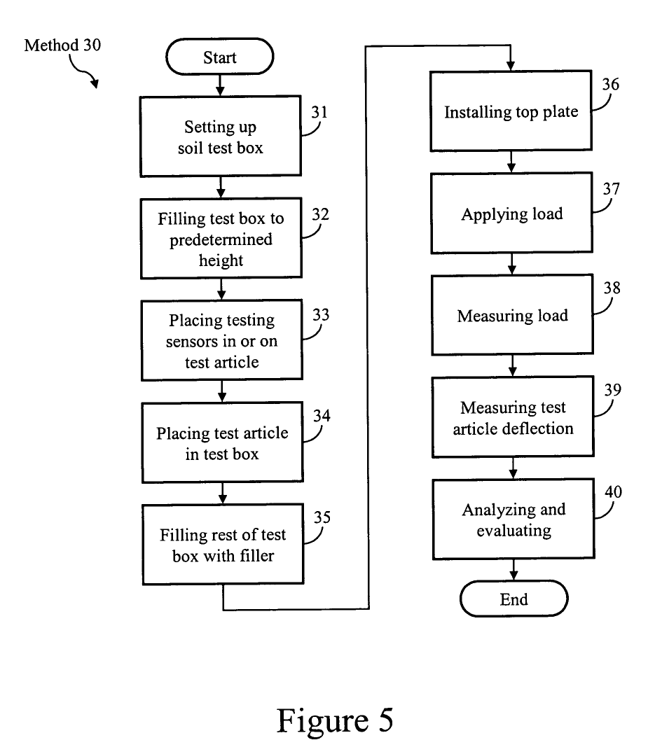

FIG. 5 shows a further flow chart illustrating in more detail a method 30 of using the flexible miniature soil test box according to another embodiment of the invention. This method includes the following steps.

Step 31—Setting up soil testing box. In this step, test box 1 is set up with a predetermined number of strips of spring steel 8 on the movable end plates 7. Strips of spring steel 8 have known properties and are adjustable so that pressure from soil compaction can be monitored and controlled. When the test article 13 is placed into the test box and a load placed on the top, the compacting soil creates a vertical pressure, which in turn is resolved into a horizontal pressure. In actual use conditions, i.e. with a pipe buried in soil, the pipe flexes to varying degrees vertically and horizontally. The soil's response to the pipe's lateral deflection needs to be simulated by the end plates. If the sides of the test box were fixed, there would be minimal deflection of the test article, and pseudo-installed conditions may not accurately be achieved.

Step 32—Filling test box to predetermined height. In this step, the test box is filled to a predetermined height with filler 15, which again could include any combination of soil, sand, gravel, or any other suitable material.

Step 33—Placing testing sensors in or on test article. In this step, sensors are placed in or on the test article 13 (pipe, column, footing, etc.). These sensors measure such things as load bearing capacity, deflection and the like, and record that data in a data log for later analysis.

Step 34—Placing test article in test box. In this step, the test article is placed into the test box on the initial bed of filler 15.

Step 35—Filling rest of test box with filler. In this step, the remaining predetermined amount of filler 15 is placed into test box over the test article. This ensures that there is filler above and below the test article, which more closely reflects installed conditions.

Step 36—Installing top plate. In this optional step, top plate 14 is placed on top of the test box. Top plate 14 has dimensions such that it can compress the contents of the test box when a load is applied from above.

Step 37—Applying load. In this step, a weight or load-applying machine, such as a Universal testing machine, applies a load on top of the test box. Typically, the applied load is between about 20 and 30 tons, but the weight of the load depends on the article being tested and the nature of the particular test.

Step 38—Measuring load. In this step, the load applied to the test box is measured to determine if the correct load has been applied, and to factor into the final test data results.

Step 39—Measuring test article deflection. In this optional step, the deflection of the test article is measured. This is a measurement of the movement of the test article as a result of compaction of the filler. Determining movement of the test article is important to define in-situ performance.

Step 39b—Measuring test article strain. In this optional step, the strain of test article 13 is measured. This is a measurement of the deformation of the test article as a result of compaction of the filler, and is also important to reflect in-situ performance.

Step 40—Analysing and evaluating. In this step, the test data derived from the sensors placed on the test article in step 33 is examined, analyzed, and evaluated to determine if the product being tested is fit for the intended purpose under the simulated environmental conditions.

It should be noted that while the preferred form of the invention has two opposing movable end walls, which is particularly suited to simulating conditions for pipes buried in open locations, many other variations of the general concept of a flexible side wall are also contemplated. For example, where it is desired to test pipes that are to be buried in a trench closely adjacent a footing or subterranean rock shelf, it may be appropriate to have only one side wall movable and the other fixed.

It should also be noted that the apparatus could similarly have more movable or deformable side walls. This may be applicable where the article to be tested is expected to deform in other ways such as may be expected with singular hollow items that may be, for example, generally spherical or cuboid in shape.

The end plates 7 as described above are not mechanically fixed to the frame. Rather, the plates rely on close fit tolerances on all sides. However, in an alternative embodiment, rollers or tracks could be used to allow for movement of the side plates 7. Furthermore, although leaf springs are described as the primary source of resilient restraint, any suitable spring device such as helical springs, pneumatic springs, hydraulic cylinders, electromechanical resistances, deformable plates or bladders, could also be employed.

The invention also contemplates numerous other variations whereby, for example, the flexible part of the side portion is configured to permit predetermined flexing under application of the load by selection of a resilient material that has these inherent properties, rather than using a rigid end plate that is mounted for controlled movement against an externally applied resilient bias force. In one such embodiment, the plates may be fixed but deformable based on the structure and/or materials from which they are made. Thus, the design of the fixed end plates would be based on material selection and geometry. For example, aluminum could be employed with an appropriate moment of inertia and thickness to provide an equivalent solution.

It will be appreciated that the flexible wall soil box of the invention has very significant advantages over the prior art. For example, the preferred embodiment substantially as illustrated was used to test a pipe having an outside diameter of about 430 mm and a length of 300 mm in a test box having a width of about 789 mm as measured between the plates 7 and a height of about 775 mm. Given that the prior art test facilities previously used for testing this sized article typically had external dimensions of at least 2000 mm by 2000 mm by 1600 mm, it can be seen that the volumetric differences are significant.

One significant advantage that flows from this is that the invention enables numerous samples to be tested simultaneously. Further, the time for setting up the tests are significantly reduced, not only by the fact that the test can be performed in-house, but because there is so much less filler material to be used and handled during the testing process. Consequently, there are significant cumulative cost savings in terms of the test equipment itself, the amount of space occupied by the test equipment, and the time involved in conducting comparable tests.

Although the invention has been described with reference to specific examples, it will be appreciated by those skilled in the art that the invention may be embodied in many other forms.

1. A soil test apparatus for testing products in subterranean soil installations, said apparatus comprising:
a container for receiving soil and a product to be buried therein for testing;
said container having a base, sides and a top opening adapted directly or indirectly to receive a load;
wherein at least one of said sides is configured to permit some movement or deformation under application of said load to the soil in the container so as to simulate larger scale installation conditions.

2. A soil test apparatus according to claim 1, wherein the container is generally rectangular in plan, having two spaced apart side walls and two mutually opposing end walls, wherein at least two of said side walls and said end walls are configured to permit movement or deformation under the application of said load to said soil.

3. A soil test apparatus according to claim 2, wherein at least one of said side walls and said end walls is configured for resilient deformation in response to pressure transferred from compacted soil in the container.

4. A soil test apparatus according to claim 2, wherein at least one of said side walls and said end walls is substantially rigid and configured for outward movement against resilient biasing means in response to pressure transferred from compacted soil in the container.

5. A soil test apparatus according to claim 4, wherein said side walls are substantially rigid, and said end walls are configured for resilient deformation or displacement in response to the applied load.

6. A soil test apparatus according to claim 4, wherein the resilient biasing means include a leaf spring arrangement secured to a fixed frame forming part of the container.

7. A soil test apparatus according to claim 1, further comprising at least one viewing window formed in one of the sides of the container.

8. A soil test the apparatus according to claim 7, wherein at least two of said sides respectively includes one of said viewing windows.

9. A soil test apparatus according to claim 1, wherein at least one of said sides includes an access port positioned to facilitate access to the product during testing.

10. A soil test apparatus according to claim 9, wherein said access port is configured to permit remote access to measurement or data recordal apparatus.

11. A soil test apparatus according to claim 1, further comprising water introduction means to permit selective variation of moisture content in the soil during testing.

12. A soil test apparatus according to claim 1, further comprising heating means to permit a measure of selective temperature variation in the soil during testing.

13. A soil test apparatus according to claim 1, further comprising cooling means to permit a measure of selective temperature variation in the soil during testing.

14. A soil test apparatus according to claim 1, being adapted for testing pipe sections to simulate subterranean pipeline installations.

15. A soil test apparatus according to claim 14, wherein the side walls in use extend generally parallel to a longitudinal axis of the pipe section and the end walls extend generally transversely to said longitudinal axis.

16. A soil test apparatus according to claim 14, wherein the side walls in use extend generally transversely to a longitudinal axis of the pipe section and the end walls extend generally parallel to said longitudinal axis.

17. A soil test apparatus according to claim 1, further comprising a lining member to minimise friction between the soil and the container.

18. A method of testing products in subterranean soil installations, said method comprising:
providing a container for receiving soil and a product to be buried therein for testing, said container having a base, sides and a top opening adapted directly or indirectly to receive a load, wherein at least one of said sides is configured to permit some movement or deformation under application of said load to the soil in the container so as to simulate larger scale installation conditions;
setting up the apparatus to permit some movement or deformation of at least one of the sides under predetermined load conditions;
partially filling the container with said soil;
positioning the product within the container;
burying the product by the addition of further soil;
applying said load to the soil via the top opening of the container; and
measuring an effect of the load on the product, the container, or the soil.

19. A method according to claim 18, wherein said measuring step includes measuring or monitoring, either continuously or intermittently, a parameter selected from the group consisting of stress or strain in the product; deformation, deflection, stress or strain in one or more of the sides of the container; compression, movement or pressure in the soil; and visual monitoring of the product.

20. A method according to claim 18, further comprising lining the container with a lining member, to minimise friction between the soil and the container.

21. A method according to claim 20, further comprising greasing the lining member.
